(19) United States
(12) Patent Application Publication
(10) Pub. No.: US 2018/0092516 A1
(43) Pub. Date: Apr. 5, 2018

(54) IMAGING DEVICE, ENDOSCOPE APPARATUS, AND IMAGING METHOD

(71) Applicant: OLYMPUS CORPORATION, Tokyo (JP)

(72) Inventor: Shinichi IMADE, Iruma-shi (JP)

(73) Assignee: OLYMPUS CORPORATION, Tokyo (JP)

(21) Appl. No.: 15/827,473

(22) Filed: Nov. 30, 2017

Related U.S. Application Data
(63) Continuation of application No. PCT/JP2015/066077, filed on Jun. 3, 2015.

(51) Int. Cl. A61B 1/00; A61B 1/04

(52) U.S. Cl. CPC A61B 1/00193; A61B 1/04; H04N 13/0257; H04N 13/0296; H04N 13/0225

(57) ABSTRACT

An imaging device includes: an image sensor; an imaging optical device; a fixed mask; and a movable mask. The imaging optical device forms an image of a subject on the image sensor. The fixed mask includes first to third openings that divide a pupil of the imaging optical device, a first filter transmitting light in a first wavelength band, a second filter transmitting light in a second wavelength band different from the first wavelength band. The movable mask includes a light shielding section and fourth to sixth openings that are provided on the light shielding section and correspond to the first to the third openings, and is movable relative to the imaging optical device. The first filter is provided to the first opening. The second filter is provided to the second opening. The third opening is provided to an optical axis of the imaging optical device.

### CROSS REFERENCE TO RELATED APPLICATION

This application is a continuation of International Patent Application No. PCT/JP2015/066077, having an international filing date of Jun. 3, 2015, which designated the United States, the entirety of which is incorporated herein by reference.

### BACKGROUND

Techniques for optically measuring a three-dimensional shape have conventionally been known, with various methods for the measuring proposed. The proposed methods include: stereoscopic imaging based on a stereoscopic view with both left and right eyes; phase shift by patterned illumination using a sinusoidal pattern and the like; and Time of Flight (TOF) based on time measurement for reflected light.

The stereoscopic imaging can be achieved with a simple mechanism with a stereoscopic optical system used for an imaging system, and thus requires no special illumination mechanisms or illumination control, and also requires no advanced signal processing. Thus, this technique can be suitably implemented in a small space and thus is advantageous in an imaging system that has been progressively downsized recently. For example, the technique can be applied to an end of an endoscope apparatus, to a visual sensor in a small robot, and for various other needs. Such an application is likely to require not only a highly accurate measurement function but also a normal observation function with high image quality. Thus, to ensure a sufficient resolution, it is a common practice to form parallax images on a common image sensor instead of using separate image sensors. The basic idea of the stereoscopic imaging is to obtain a distance to a subject based on an amount of parallax between left and right images. If the left and right images fail to be separately formed on the common image sensor, the amount of parallax cannot be detected, and thus the distance information cannot be obtained.

JP-A-2010-128354 discloses an example of a method of separately forming left and right images. Specifically, switching between left and right imaging optical paths is performed along time with a mechanical shutter, so that the left and the right images are obtained in a time-division manner JP-A-2013-3159 discloses another method in this context. Specifically, an RG filter is inserted in the left half of a single imaging optical path, and a GB filter is inserted in the right half of the path, so that left and right images are separately obtained based on an R image and a B image in a captured image. In JP-A-2013-3159, an observation image is acquired in a normal observation mode, with the RG filter and the GB filter retracted from the imaging optical path.

### SUMMARY

According to one aspect of the invention, there is provided an imaging device comprising: an image sensor;

an imaging optical device forming an image of a subject on the image sensor;

a fixed mask including first to third openings dividing a pupil of the imaging optical system, a first filter transmitting light in a first wavelength band, and a second filter transmitting light in a second wavelength band different from the first wavelength band;

a movable mask including a light shielding section and fourth to sixth openings that are provided on the light shielding section and correspond to the first to the third openings, the movable mask being movable relative to the imaging optical system,

the first filter is provided to the first opening,

the second filter being provided to the second opening,

the third opening being provided on an optical axis of the imaging optical device.

According to another aspect of the invention, there is provided an imaging device comprising: an image sensor;

an imaging optical device forming an image of a subject on the image sensor;

a fixed mask including first to third openings dividing a pupil of the imaging optical system, a first filter transmitting light in a first wavelength band, and a second filter transmitting light in a second wavelength band different from the first wavelength band;

a movable mask including a light shielding section, a fourth opening that is provided on the light shielding section and corresponds to the first to the third openings, and a fifth opening that is provided on the light shielding section and corresponds to the second opening, the movable mask being movable relative to the imaging optical system,

the first filter is provided to the first opening,

the second filter being provided to the second opening,

the third opening being provided on an optical axis of the imaging optical device.

According to another aspect of the invention, there is provided an imaging device comprising: an image sensor;

an imaging optical device forming an image of a subject on the image sensor;

a movable mask including first to the third openings, the movable mask being movable relative to the imaging optical system;

a fixed mask including a fourth opening provided on an optical axis of the imaging optical device; and

a processor being configured to implement a movable mask control process for controlling the movable mask,

the movable mask includes:

a first filter being provided to the first opening and transmitting light in a first wavelength band; and

a second filter being provided to the second opening and transmitting light in a second wavelength band different from the first wavelength band,

the fourth opening has

a size larger than a distance between the first and the second openings,

the processor implementing the movable mask control process including:

setting, in a non-stereoscopic mode, the movable mask to be in a first state in which the first and the second openings do not overlap with the fourth opening and the third opening is on the optical axis, as viewed in a direction of the optical axis; and

setting, in a stereoscopic mode, the movable mask to be in a second state in which the first and the second openings overlap with the fourth opening, and the third opening does not overlap with the fourth opening, as viewed in the direction of the optical axis.

According to another aspect of the invention, there is provided an endoscope apparatus comprising the imaging device as defined in any of the above.

According to another aspect of the invention, there is provided an imaging method comprising: setting, in a non-stereoscopic mode, a movable mask, including a light shielding section and fourth to sixth openings that are provided to the light shielding section and correspond to first to third openings of a fixed mask, to be in a first state, in such a manner that the light shielding section overlaps with the first opening provided with a first filter transmitting light in a first wavelength band and the second opening provided with a second filter transmitting light in a second wavelength band different from the first wavelength band, and that the sixth opening overlaps with the third opening, as viewed in a direction of an optical axis of an imaging optical device; and

setting, in a stereoscopic mode, the movable mask to be in a second state in such a manner that the fourth and the fifth openings overlap with the first and the second openings and the light shielding section overlaps with the third opening, as viewed in the direction of the optical axis.

### BRIEF DESCRIPTION OF THE DRAWINGS

FIG. 1 illustrates an example of a basic configuration according to one embodiment.

FIG. 2 further illustrates the example of the basic configuration according to the embodiment.

FIG. 3 illustrates an example of a detailed configuration of a fixed mask and a movable mask.

FIG. 4 further illustrates the example of the detailed configuration of the fixed mask and the movable mask.

FIG. 5 illustrates spectral characteristics of pupils.

FIG. 6 illustrates a first modification of the fixed mask and the movable mask.

FIG. 7 illustrates the first modification of the fixed mask and the movable mask.

FIG. 8 illustrates a second modification of the fixed mask and the movable mask.

FIG. 9 illustrates the second modification of the fixed mask and the movable mask.

FIG. 10 illustrates the principle of stereoscopic measurement.

FIG. 11 illustrates a configuration example of an endoscope apparatus according to the embodiment.

FIG. 12 illustrates a sequence of switching between an observation mode and a stereoscopic measurement mode.

### DESCRIPTION OF EXEMPLARY EMBODIMENTS

Stereoscopic imaging described above is negatively affected by a movement of an imaging system or a subject. For example, in JP-A-2010-128354, right and left images are captured in a time-division manner. Thus, the movement of the imaging system or the subject results in detection of a phase difference including a shifted amount due to the movement. This results in a measurement error because the phase difference is difficult to be separated into the shifted amount and the actual phase difference. In JP-A-2013-3159, image capturing is switched between that for an observation image and that for a parallax image. However, this technique is designed for autofocusing, and is not designed for high speed switching required for an observation image and a parallax image to match in the three-dimensional shape measurement. The configuration in JP-A-2013-3159 includes two movable sections, and thus requires a large driving mechanism and involves a higher risk of failure or other like disadvantages.

For example, in an application, such as an endoscope apparatus, where a camera is not fixed relative to a subject, the relative movement between the imaging system and the subject is likely to have a negative impact. In other words, if the movement can occur without imposing any negative impact, shape measurement with a moving camera or other like measurement, which has been difficult in the conventional techniques, might be achievable.

Some aspects of the present embodiment can provide an imaging device, an endoscope apparatus, an imaging method, and the like with which stereoscopic measurement can be performed and an observation image can be captured while being less affected by the movement of the imaging system or a subject.

According to another embodiment of the invention, there is provided an imaging device comprising: an image sensor;

an imaging optical device forming an image of a subject on the image sensor;

a fixed mask including first to third openings dividing a pupil of the imaging optical system, a first filter transmitting light in a first wavelength band, and a second filter transmitting light in a second wavelength band different from the first wavelength band;

a movable mask including a light shielding section and fourth to sixth openings that are provided on the light shielding section and correspond to the first to the third openings, the movable mask being movable relative to the imaging optical system,

the first filter is provided to the first opening,

the second filter being provided to the second opening,

the third opening being provided on an optical axis of the imaging optical device.

According to one aspect of the present embodiment, the movable mask is movable relative to the imaging optical device. The stereoscopic measurement can be performed and an observation image can be captured with the position of the movable mask switched. For example, two optical paths are provided so that stereoscopic imagine can be performed in a non-time-division manner, and a single movable mask is used as the movable section. Thus, the negative impact due to the movement of the imaging system or the subject can be reduced.

The present embodiment will be described below. The present embodiment described below does not unduly limit the scope of the present invention described in the appended claims. Not all the components described in the present embodiment are required to embody the present invention.

In the description below, an example where the present invention is applied to an industrial endoscope apparatus is described. However, the application of the present invention is not limited to industrial endoscope apparatuses. The present invention may be applied to any three-dimensional measurement device that measures a three-dimensional shape through stereoscopic imaging (a method of acquiring distance information on a subject by detecting a phase difference between two images obtained with an imaging system involving parallax), and to any imaging device having a three-dimensional measurement function (such as a medical endoscope apparatus, a microscope, an industrial camera, and a visual function of a robot, for example).

1. Basic Configuration

First of all, an overview of the present embodiment is described, and then a basic configuration (principle configuration) according to one embodiment is described.

For example, an examination using an endoscope apparatus is performed as follows. A scope is inserted into an examination target to check whether there is an abnormality while capturing normal images. When a portion, such as a scar, to be observed in detail is found, the three-dimensional shape of the portion is measured to determine whether a further examination is required. Thus, the normal observation image is captured with white light. For example, stereoscopic imaging may be performed with white light so that stereoscopic measurement and the image capturing with white light can both be achieved. The stereoscopic imaging using white light requires an image sensor to be divided into left and right regions, and a left image and a right image to be respectively formed on the left and the right regions. Thus, only an image with a low resolution can be obtained. A color phase difference method may be employed to form the left and the right images on a single region of the image sensor. Unfortunately, this method results in a captured image with color misregistration that is unacceptable as the observation image.

In view of the above, time-division switching (for example, JP-A-2010-128354) is required for forming the left and the right images on the single region of the image sensor with white light. However, relative movement between an imaging system and a subject leads to shifting due to the movement between the left and the right images, resulting in inaccurate triangulation. Devices such as endoscope cannot have a camera fixed relative to the subject and thus are highly likely to involve this shifting due to movement.

In the present embodiment, an observation image with high resolution can be captured with white light, and the stereoscopic measurement in a non-time-division manner can be performed based on the color phase difference method.

JP-A-2013-3159 described above discloses an example of performing the stereoscopic measurement in a non-time-division manner based on the color phase difference method. However, the configuration in JP-A-2013-3159 employs the stereoscopic measurement for autofocusing, and thus it is reasonable to believe that the configuration is not designed for high speed switching between the mode for observation image and the mode for stereoscopic measurement. Furthermore, the configuration includes two filters as movable sections, and thus is unsuitable for the high speed switching in the first place.

Furthermore, in the configuration in JP-A-2013-3159, a single optical path is simply divided into left and right sides at the center, and thus is difficult to ensure a sufficient distance between pupils. Thus, accuracy of the distance measurement is difficult to improve. The endoscope apparatus needs to perform panning and focusing, and thus has a small aperture stop (large F value). Logically, dividing a small diameter of such an aperture stop into left and right sides is likely to result in a short distance between the pupils.

The time-division switching, including time-division switching between left and right for stereoscopic imaging, requires mechanical (switching) motion of a shutter and a spectral filter. The mechanical motion inherently involves a risk of error and failure, and the switched states (positions) of the shutter and the spectral filter need to be detected, and correction is required when an error is found. When such a detection function is implemented, the detection and correction are easier with a smaller variety of errors involved. In this context, the configuration in JP-A-2013-3159 is difficult to guarantee the detection and the correction because the configuration involves a risk of various types of errors and example of which includes one or both of the two spectral filters failing to be inserted in the pupil.

The present embodiment can overcome these problems described above with the following configuration. More specifically, in a single optical system, a pupil center, a left pupil, and a right pupil area separately provided, and images formed with the pupils are formed on a common region of a single image sensor. The configuration includes a switching mechanism so that high speed alternate switching between left-pupil and right-pupil optical paths (first optical path, second optical path) and a pupil-center optical path (third optical path) can be achieved, and performs time-division switching between an observation mode, in which a first image (observation image) is acquired, and a measurement mode, in which a second image (parallax image, stereoscopic image, left and right images, or measurement image) is acquired.

The switching mechanism is set in such a manner that the first image, used for a normal observation, is obtained only with the pupil-center optical path, and that the second image, used for measurement, is obtained with images formed with the left-pupil optical path and the right-pupil optical path overlapped with each other. Spectral filters are provided in the optical paths so that the left-eye image and the right-eye image correspond to separate wavelength bands.

Generally, the observation image is a normal color image involving no parallax, whereas the measurement image includes left and right separate parallax images. Three-dimensional information is acquired by obtaining an amount of parallax by using separate images, and then calculating distance information, indicating a distance to the subject, based on a principle of the stereoscopic measurement. In the measurement mode, the parallax images can be simultaneously obtained, and thus the system is free of a measurement error factor due to movement of the subject or the imaging system. As described later, the imaging system with the left-eye optical path and the right-eye optical path as separate paths can be implemented with a single movable section, and thus can achieve high speed switching, smaller size, error detection, and the like. The imaging system with the left-pupil optical path and the right-pupil optical path as separate paths can be downsized while ensuring the parallax, and can achieve higher measurement accuracy.

An application of the present invention includes a device having an imaging system that is not stably positioned (fixed) and having an imaging mechanism too small to use a large image sensor for ensuring a sufficient resolution. A typical example of such a device includes an industrial endoscope. Still the application of the present invention is not limited to such a device, and the present invention can be widely applied to a three-dimensional measurement device directed to high-resolution monitoring and highly accurate measurement.

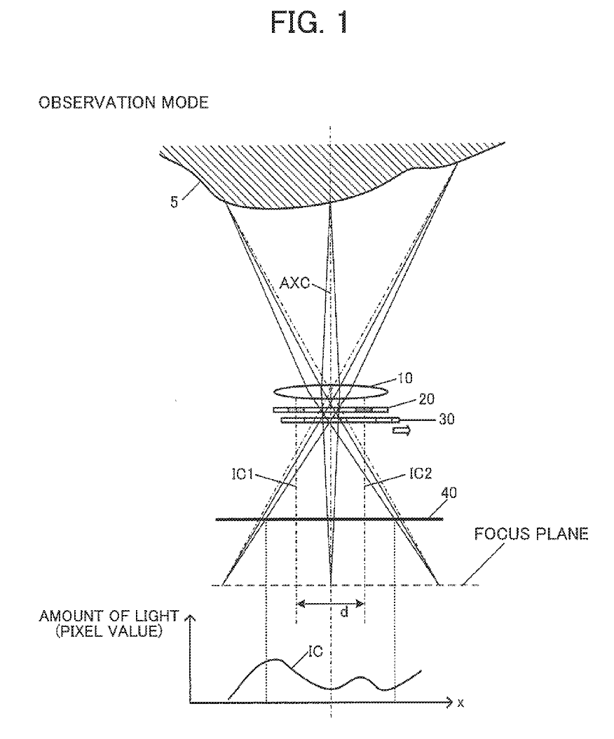

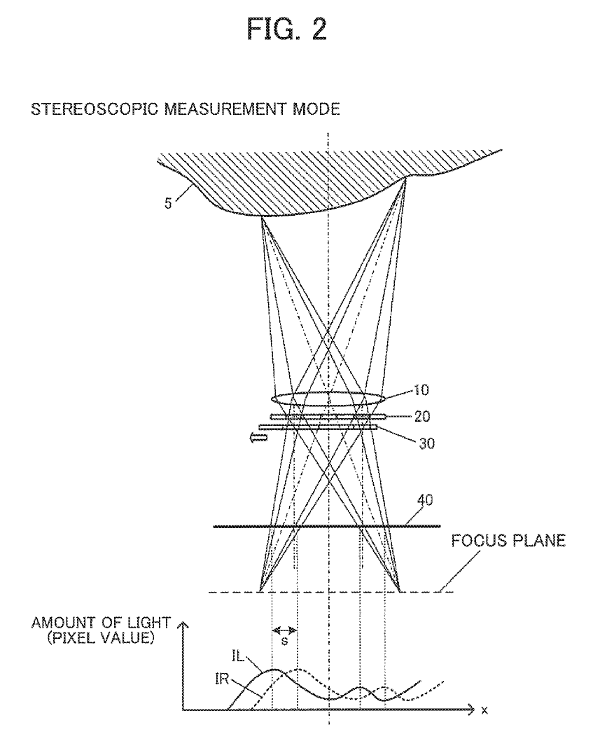

Now, the basic configuration according to the present embodiment is described with reference to FIG. 1 and FIG. 2. FIG. 1 and FIG. 2 each include a cross-sectional view of an image capturing section as viewed in a lateral direction (on a plane including an optical axis) and a graph illustrating a relationship between an amount of light focused on the image sensor (or a pixel value of an image formed on the image sensor) and a position x. The position x is a position (coordinate) in a direction orthogonal to the optical axis of the imaging optical system, and is a pixel position of the image sensor for example. Although the position is actually defined in a two-dimensional coordinate system, the position is described based on a one-dimensional coordinate system corresponding to a parallax direction in the two dimensional coordinate system.

The endoscope apparatus according to the present embodiment includes: an imaging optical system 10; a movable mask 30 (first mask); a fixed mask 20 (second mask); and an image sensor 40. The imaging optical system 10 is a single optical system including one or a plurality of lenses. In the example described herein, the image sensor 40 includes a color filter with RGB Bayer arrangement. However, this should not be construed in a limiting sense. For example, a complementary color filter or the like may be provided.

As illustrated in FIG. 1 and FIG. 2, reflected light from a subject 5 passes through the imaging optical system 10 so that an image is formed on the image sensor 40 based on the light. The fixed mask 20 divides the pupil into the pupil center and left and right pupils, and the movable mask 30 switches between image forming with the pupil center and image forming with the left and the right pupils. The images are formed in the same region of the image sensor 40. A light emitting mechanism that emits light onto the subject 5 is omitted in the figure. In the figure, d represents a distance between a centerline IC1 of the left pupil (left-eye stop hole of the fixed mask 20) and a centerline IC2 of the right pupil (right-eye stop hole of the fixed mask 20), serving as a baseline length in the stereoscopic measurement. A straight line AXC represents an optical axis of the imaging optical system 10. For example, the centerlines IC1 and IC2 are disposed at an equal distance from the optical axis AXC of the single imaging optical system 10. The centerlines IC1 and IC2 and the optical axis AXC are preferably in the same plane but do not necessarily need to be in the same plane.

For example, the fixed mask 20 and the movable mask 30 are disposed at a pupil position of the imaging optical system 10, and may be disposed more on the image side than the imaging optical system 10. The fixed mask 20 is fixed with respect to the imaging optical system 10, whereas the movable mask 30 can have the position switched on a plane orthogonal to the optical axes AXC. Thus, the movable mask 30 can be in a first state illustrated in FIG. 1, corresponding to the observation mode (first mode, non-stereoscopic mode, a single optical system mode), and in a second state, corresponding to a stereoscopic measurement mode (second mode, stereoscopic mode) illustrated in FIG. 2. These two modes can be switched from one to another at high speed.

The fixed mask 20 includes: a light shielding section (light shielding member) including three stop holes (a left-eye stop hole, a right-eye stop hole, and a center stop hole); a short-wavelength (blue) spectral filter provided to the left-eye stop hole; and a long-wavelength (red) spectral filter provided to the right-eye stop hole. A portion other than the stop hole is covered with the light shielding section, and thus light cannot pass through this portion. For example, the center stop hole may be a through hole, or may be provided with a spectral filter of some sort (for example, a broadband spectral filter at least transmitting white light).

The movable mask 30 includes a light shielding section (light shielding member) that has a plate shape and is provided with three stop holes. The size of the movable mask 30 is set in such a manner that the center stop hole or the left and right stop holes of the three stop holes of the fixed mask 20 can be covered in each mode. The stop holes are provided at a position overlapping with the center stop hole of the fixed mask 20 in the observation mode, and at a position overlapping with the left-eye stop hole and the right-eye stop hole in the stereoscopic measurement mode. The stop holes of the movable mask 30 are hereinafter also referred to as the left-eye stop hole, the right-eye stop hole, and the center stop hole. FIG. 1 and FIG. 2 each illustrate a configuration where the movable mask 30 is disposed more on the image side than the fixed mask 20. Alternatively, the movable mask 30 may be disposed more on the object side than the fixed mask 20.

The spectral characteristics of the left-eye stop hole, the right-eye stop hole, and the center stop hole of the fixed mask 20 are hereinafter respectively denoted with FL, FR, and FC, the spectral characteristics of the left-eye stop hole, the right-eye stop hole, and the center stop hole of the movable mask 30 are hereinafter respectively denoted with SL, SR, and SC. For the sake of understanding, the spectral filters provided to the stop holes are also denoted with the signs FL, FR, FC, SL, SR, and SC.

FIG. 1 illustrates the state corresponding to the observation mode. In this state, the optical path corresponding to the pupil center (pupil-center optical path) is in the open state through the center stop hole of the fixed mask 20 and the center stop hole of the movable mask, and the optical paths corresponding to the left and the right pupils (left- and right-pupil optical paths) are in a blocked (shielded) state by the movable mask 30. Thus, an image IL formed on the image sensor 40 is obtained with the pupil center only, whereby a normal captured image (obtained with a single optical system and white light) is obtained.

FIG. 2 illustrates the state corresponding to the stereoscopic measurement mode. In this state, the left-eye stop hole of the fixed mask 20 and the left-eye stop hole of the movable mask 30 overlap with each other, and the right-eye stop hole of the fixed mask 20 and the right-eye stop hole of the movable mask 30 overlap with each other. The pupil-center optical path is closed (shielded) by the movable mask 30. Thus, in the left-pupil optical path, light for image forming is filtered by the short-wavelength (blue) spectral filter SL (first filter). Thus, an image IL′ including the resultant short wavelength components is formed on the image sensor 40. In the right-pupil optical path, the light for image forming is filtered by the long-wavelength (red) spectral filter FR (second filter). Thus, an image IR′ including long-wavelength components is formed on the same image sensor 40.

In this manner, in the stereoscopic measurement mode, the image IL′, which is the short-wavelength image obtained with blue pixels in the image sensor 40, and the image IR′, which is the long-wavelength image obtained with red pixels in the image sensor 40, can be separately acquired through the two optical paths. Thus, in the stereoscopic measurement mode, the left-eye image IL′ and the right-eye image IR′, with a phase difference, can be simultaneously and separately obtained, whereby the stereoscopic measurement can be performed with the phase difference images.

2. Fixed Mask and Movable Mask

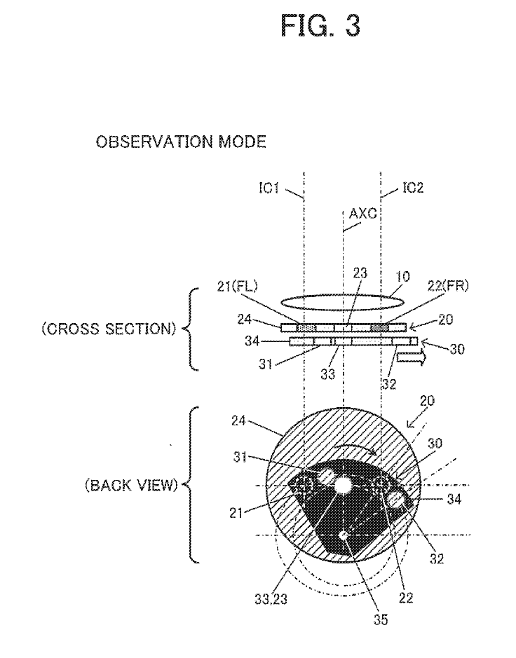

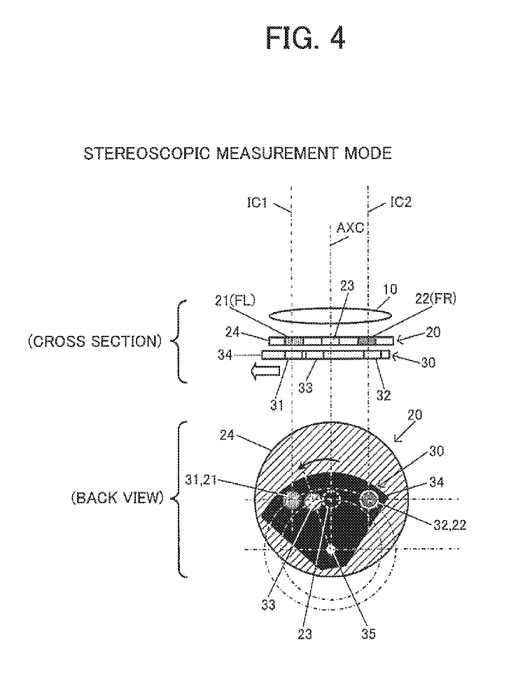

FIG. 3 and FIG. 4 illustrate detail configuration examples of the fixed mask 20 and the movable mask 30. FIG. 3 and FIG. 4 each include a cross-sectional view of the imaging optical system 10, the fixed mask 20, and the movable mask 30, and a diagram illustrating the fixed mask 20 and the movable mask 30 as viewed in the optical axis direction (a back view as viewed from the image side).

The left-pupil optical path of the fixed mask 20 has a stop hole 21 provided with the short-wavelength spectral filter FL. The right-pupil optical path has a stop hole 22 provided with the long-wavelength spectral filter FR. The pupil-center optical path is provided with a stop hole 23 in an open state (through hole). The stop holes 21 and 22 are holes with sizes corresponding to the depth of field required for the imaging system for example (for example, circular holes with a size defined with a diameter), and are formed in a light shielding section 24 (light shielding member). The stop holes 21, 22, and 23 have the centers (the center of a circle for example) respectively matching (or substantially matching) the centerlines IC1 and IC2 and the optical axis AXC. The light shielding section 24 is a plate-shaped member provided to be orthogonal with respect to the optical axis AXC for example, to shield a casing, including the imaging optical system 10, in front view (or back view) of the casing.

The movable mask 30 includes: open-state (through-hole) stop holes 31, 32, and 33; and a light shielding section 34 (light shielding member) provided with the stop holes 31, 32, and 33. The stop holes 31, 32, and 33 are slightly larger than the stop holes 21, 22, and 23 of the fixed mask 20, for example, or may be a hole with a size corresponding to the depth of field required for the imaging system (for example, a circular hole with a size defined by a diameter). In the stereoscopic observation mode, the stop hole 33 has the center (for example, the center of the circle) matching (or substantially matching) the optical axis AXC. The light shielding section 34 is connected to a rotational shaft 35 orthogonal to the optical axis AXC, and is a plate-shaped member provided to be orthogonal to the optical axis AXC for example. The light shielding section 34 has a form of a fan (with a pointed end of the fan connected to the shaft 35). However, this should not be construed in a limiting sense, and any shape may be employed as long as the states illustrated in FIG. 3 and FIG. 4 can be achieved.

The movable mask 30 rotates about the rotational shaft 35 by a predetermined angle in the direction orthogonal to the optical axis AXC. For example, this rotational motion can be implemented with a piezoelectric element, a motor, or the like. In the observation mode illustrated in FIG. 3, the pupil-center optical path (stop hole 23) of the fixed mask 20 is in the open state and the left- and the right-pupil optical paths (stop holes 21 and 22) are in the shielded state, as a result of the rotation and inclination of the movable mask 30 toward the right-eye side by the predetermined angle. In the stereoscopic measurement mode illustrated in FIG. 4, the pupil-center optical path (stop hole 23) of the fixed mask 20 is in the shielded state and the left- and the right-pupil optical paths (stop holes 21 and 22) are in the open state, as a result of the rotation and inclination of the movable mask 30 toward the left-eye side by the predetermined angle. The stop hole 21 provided with the spectral filter FL is exposed so that only the short-wavelength components can pass through the left pupil. The stop hole 22 provided with the spectral filter FR is exposed so that only the long-wavelength components can pass through the right pupil.

In the description above, the two states are established with the movable mask 30 rotated by the predetermined angle about the shaft. However, this should not be construed in a limiting sense. For example, the two states may be established with a sliding motion of the movable mask 30. For example, the rotational motion or the sliding motion can be implemented with a magnet mechanism, a piezoelectric mechanism, or the like that may be appropriately selected to achieve a high speed motion and high resistance.

3. Spectral Characteristics of Pupils

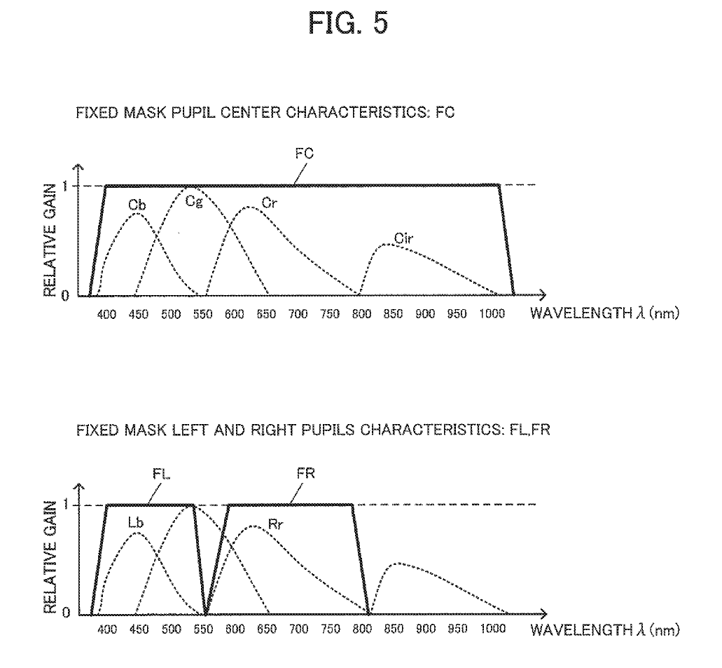

FIG. 5 illustrates the spectral characteristics FC, FL, and FR of the pupil-center optical axis, the left-eye optical axis, and the right-eye optical axis of the fixed mask 20. In FIG. 5, a relative gain represents relationship between a transmittable wavelength and a transmittance of the spectral filter (or the through hole). A dotted line represents the spectral characteristics (spectral sensitivity characteristics) of color pixels in the image sensor 40, as reference characteristics. Signs “C”, “L”, and “R” respectively represent the pupil-center optical path, the left-eye optical path, and the right-eye optical path. Signs “r”, “g”, “b”, and “ir” respectively represent a red color, a green color, a blue color, and near infrared. For example, “Lb” represents the spectral characteristics of light that passes through the left-pupil optical path to be detected by blue pixels of the image sensor 40. For the sake of understanding, each of images obtained with these spectral characteristics is also denoted with the corresponding sign (Lb or the like).

As illustrated in FIG. 5, the spectral characteristics FC of the pupil-center optical path of the fixed mask 20 includes all the spectral characteristics Cb, Cg, Cr, and Cir of the color pixels of the image sensor 40. The spectral characteristics may be set for the light emitted onto the subject 5 in a simple open state (through hole). Alternatively, the stop hole 23 of the pupil center may be provided with the spectral filter having the spectral characteristics FC illustrated in FIG. 5.

The spectral characteristics FL of the left-pupil optical path of the fixed mask 20 include the spectral characteristics Lb of the blue color b but do not include the spectral characteristics of the red color r. Note that the spectral characteristics FL need not to be completely different from the spectral characteristics of the red color r or may not include the spectral characteristics Lb of blue color b entirely, as long as the separation between the left and the right images (the red image and the blue image) can be sufficiently ensured.

The spectral characteristics FR of the right-pupil optical path of the fixed mask 20 include the spectral characteristics Rr of the red color r but do not include the spectral characteristics of the blue color b. Note that the spectral characteristics FR need not to be completely different from the spectral characteristics of the blue color b or may not include the spectral characteristics Rr of red color r entirely, as long as the separation between the left and the right images (the red image and the blue image) can be sufficiently ensured.

The stop holes 31, 32, and 33 of the movable mask 30 are obtained by simply achieving the open state of the stop holes 21, 22, and 23 of the fixed mask 20, and thus are not limited to particular spectral characteristics. For example, spectral characteristics that are the same as the spectral characteristics FC may be employed.

4. Captured Image

In the observation mode, the captured image is acquired only through the pupil-center optical path, and thus includes the components of the red color r, the green color g, the blue color b, and near infrared ir. Thus, an image captured with a single optical system, with no parallax image superimposed thereon, can be simply obtained.

Generally, the image sensor 40 with an RGB Bayer array has color pixels sensitive to a red component Cr, a green component Cg, and a blue component Cb. Each pixel is sensitive to a near infrared component Cir. Thus, in the observation mode, three types of color images Vr, Vg, and Vb represented by the following Formula (1) can be separately obtained. More specifically, Vr, Vg, and Vb respectively represent a red image, a green image, and a blue image (or their spectral characteristics) in the observation mode.

$$\begin{matrix}[ Formula 1 ] & \\ \begin{matrix}Vr = Lr + Lir \\ Vg = Lg + Lir \\ Vb = Lb + Lir\end{matrix} \} & ( 1 )\end{matrix}$$

In the stereoscopic measurement mode, two types of parallax images, obtained through the left-pupil optical path and the right-pupil optical path, are formed on the same image sensor 40 while being overlapped with each other, whereby a captured image involving image shift (phase difference s in FIG. 2) is acquired. The image shift corresponds to the amount of parallax with which depth information on the subject can be obtained, according to the principle of the stereoscopic measurement. To obtain the amount of parallax, the left-eye image and the right-eye image need to be separately obtained, and the phase difference needs to be detected by checking the correlation between the images (matching).

Thus, the left-pupil optical path is set to have the spectral characteristics FL, in the stereoscopic mode, for transmitting light with a wavelength not longer than 550 nm and blocking light with a wavelength not shorter than 550 nm is blocked. The spectral characteristics FR of the right-eye optical path of the fixed mask 20 is set in such a manner that light with a wavelength not longer than 800 nm passes through and light with a wavelength not shorter than 550 nm is blocked. In any case, the spectral filters FL and FR are set in accordance with the spectral sensitivity characteristics of the blue and the red pixels of the image sensor 40.

Thus, in the stereoscopic measurement mode, the left-pupil image from the left-pupil optical path is obtained as an image with the spectral characteristics Lb obtained by the spectral characteristics of the blue pixels in the image sensor 40 (RGB Bayer array). Thus, the right-pupil image from the right-pupil optical path is obtained as an image with the spectral characteristics Lr due to the spectral characteristics of the red pixels in the image sensor 40 (RGB Bayer array). Thus, a left-pupil image Mr and a right-pupil image Mb, represented by Formula (2), can be separately obtained with color pixels different from each other. Specifically, Mr and Mb respectively represent a red image and a blue image (or their spectral characteristics) in the stereoscopic measurement mode. When the image sensor 40 supports complementary colors, complementary color information (cyan, magenta, yellow) may be converted so that the red image Mr and the blue image Mb are extracted.

$$\begin{matrix}[ Formula 2 ] & \\ \begin{matrix}Mr = Rr \\ Mb = Lb\end{matrix} \} & ( 2 )\end{matrix}$$

In the embodiment described above, an imaging device (endoscope apparatus) includes: the image sensor 40; the imaging optical system (imaging optical device) 10; the fixed mask 20; and the movable mask 30. The imaging optical system 10 forms an image of the subject 5 on the image sensor 40. The fixed mask 20 includes: the first to the third openings (the stop holes 21, 22, and 23) dividing the pupil of the imaging optical system 10; the first filter FL transmitting light in the first wavelength band; and the second filter FR transmitting light in the second wavelength band different from the first wavelength band. The movable mask 30 includes the light shielding section 34 and fourth to sixth openings (stop holes 31, 32, and 33) that are provided on the light shielding section 34 and correspond to the first to the third openings (stop holes 21, 22, and 23), and is movable relative to the imaging optical system 10. The first filter FL is provided to the first opening (stop hole 21). The second filter FR is provided to the second opening (stop hole 22). The third opening (stop hole 23) is provided to the optical axis AXC of the imaging optical system 10.

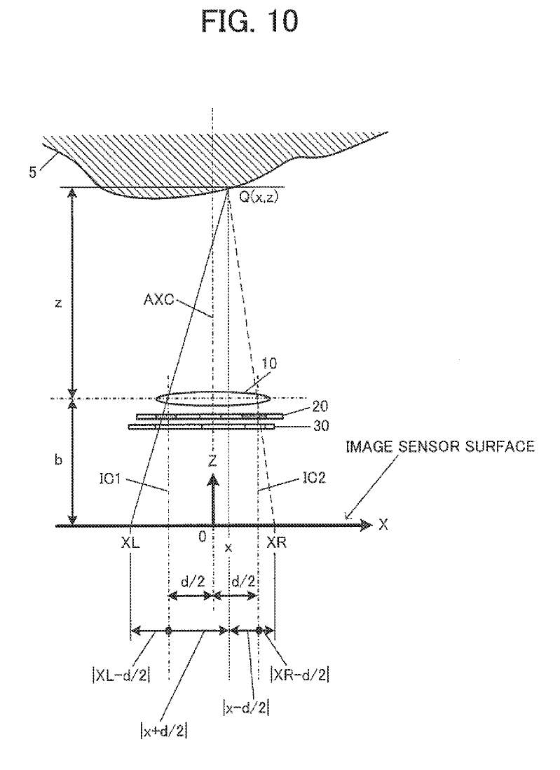

This configuration can achieve the switching between the observation mode and the stereoscopic measurement mode as described above with reference to FIG. 1 to FIG. 4. The parallax images in the color phase difference method can be simultaneously (non time-division manner) acquired, whereby stereoscopic measurement can be accurately performed. The movable mask 30 is provided as a single movable section. Thus, switching between the modes can be achieved at high speed, a simple driving mechanism, and with a less risk of failure and error. The movable mask 30 can be achieved with a simple configuration obtained with the openings (stop holes 31, 32, and 33) provided to the light shielding section 34. Thus, a risk involved in the vibration due to the switching such as detaching of the filter can be reduced. The left and the right pupils can be clearly separated from each other by the openings (aperture openings 21 and 22) of the fixed mask 20, and thus can have a long baseline length (d in FIG. 1 and FIG. 10) for the stereoscopic measurement, whereby the distance measurement can be accurately performed.

For example, in a comparative example where a single observation image is captured with one of the left and the right pupils, the image is captured with a pupil displaced relative to the optical axis. In the present embodiment, the three openings (stop holes 21, 22, and 23) are formed in the fixed mask 20 and one of the openings is provided on the optical axis AXC. Thus, the observation image is obtained with the pupil center. Thus, an observation image with a large angle of view can be obtained with only small vignetting caused by a light beam. Furthermore, a high quality (small distortion, for example) image can be formed.

Positional relationship between a position of the subject 5 and a pixel position is set in such a manner that the center (position s/2) of a phase difference (s in FIG. 2) in the stereoscopic measurement matches the light beam passing through the pupil center. Thus, in the present embodiment, matching pixels in the observation image and the distance map correspond to the same position on the subject 5. On the other hand, in the comparative example described above, different pixels in the observation image and the distance map correspond to a single position on the subject 5, because the observation image has parallax on the left side and is not obtained with the pupil center. The present embodiment is more advantageous in a configuration where the observation image is displayed with three-dimensional information overlaid thereon.

In the present embodiment, the first opening (stop hole 21) corresponds to the left pupil, the second opening (stop hole 22) corresponds to the right pupil, and the third opening (stop hole 23) corresponds to the pupil center. Alternatively, the first opening may correspond to the right pupil, and the second opening may correspond to the left pupil. The pupil is separated in the left and right direction in the stereoscopic measurement for the sake of description, but the separation direction of the pupil is not limited to the left and right direction. In the present embodiment, the openings are referred to as stop holes. Note that the openings do not necessarily have to have a function of an aperture stop (function of restricting a cross-sectional area of a bundle of rays traveling through a pupil). For example, in the observation mode, the stop holes 23 and 33 overlap. When the stop hole 23 is smaller, the stop hole 23 serves as the aperture stop. When the stop hole 33 is smaller, the stop hole 33 serves as the aperture stop.

The pupil is for separating (or defining) an imaging optical path in the imaging optical system 10. The optical path is a path through which light, corresponding to an image to be formed on the image sensor 40 and entered from an object side of the optical system, reaches the image sensor 40. Specifically, the first and the second optical paths are optical paths that pass through the imaging optical system 10 and the stop holes 21 and 22 of the fixed mask 20 (and also the stop holes 31 and 32 of the movable mask 30 in the stereoscopic measurement mode). The third optical path is an optical path that passes through the imaging optical system 10 and the stop hole 23 of the fixed mask 20 (and also the stop hole 33 of the movable mask 30 in the observation mode).

The mask is a member or a component shielding light incident on the mask and also transmitting a part of the light. The fixed mask 20 and the movable mask 30 according to the present embodiment have the light shielding sections 24 and 34 that shield the light and the stop holes 21, 22, 23, 31, 32, and 33 through which the light transmits (entire wavelength band, or a part of the entire wavelength band).

For example, in the present embodiment, the first wavelength band corresponds to the blue wavelength band (the wavelength on the shorter wavelength side of the white light). The second wavelength band corresponds to the red wavelength band (the wavelength band on the longer wavelength side of the white light). Alternatively, the first wavelength band may correspond to the red wavelength band, and the second wavelength band may correspond to the blue wavelength band. The first wavelength band and the second wavelength band may be set in any way as long as the image obtained with the first optical path and the image obtained with the second optical path can be separated from each other based on the wavelength band. In the present embodiment, the blue image and the red image are separately obtained with the Bayer image sensor. However, this should not be construed in a limiting sense. The present invention may be applied to any method with which parallax images are separately obtained based on the wavelength band.

In the present embodiment, the imaging device includes a movable mask control section 340 that controls the movable mask 30 (FIG. 13). In the non-stereoscopic mode (observation mode), the movable mask control section 340 (processor) sets the movable mask 30 to be in the first state (at a first position) in which the light shielding section 34 overlaps with the first and the second openings (stop holes 21 and 22) and the sixth opening (stop hole 33) overlaps with the third opening (stop hole 23), as viewed in the direction along the optical axis AXC. In a stereoscopic mode (stereoscopic measurement mode), the movable mask control section 340 sets the movable mask 30 to be in the second state (at a second position) in which the fourth and the fifth openings (stop holes 31 and 32) overlap with the first and the second openings (stop holes 21 and 22), and the light shielding section 34 overlaps with the third opening (stop hole 23), as viewed in the direction along the optical axis AXC.

With such driving control for the movable mask 30, control for switching between the observation mode illustrated in FIG. 1 and FIG. 3 and the stereoscopic measurement mode illustrated in FIG. 2 and FIG. 4 can be achieved. Specifically, when the movable mask 30 is set to be in the first state, the first and the second openings are shielded by the light shielding section 34, and thus an image is captured only with the third opening. Thus, a normal observation image (white light image) can be captured because no spectral filter is inserted in the third opening. When the movable mask 30 is set to be in the second state, the first filter FL is fixed to the first opening and the second filter FR is fixed to the second filter, and thus parallax images in the color phase difference method can be captured.

In the present embodiment, the captured image obtained with the image sensor 40 includes images of the red color r, the green color g, and the blue color b. The first wavelength band FL corresponds to one of the red color r and the blue color b. The second wavelength band FR corresponds to the other one of the red color r and the blue color b.

For example, in the present embodiment, the first wavelength band FL is a blue wavelength band (the band SL corresponding to the characteristics Lb in FIG. 5). The second wavelength band FR is the red wavelength band (the band FR corresponding to the characteristics Rr in FIG. 5). However, this should not be construed in a limiting sense, and the opposite combination between the bands and the colors may be employed The first wavelength band FL may include at least a part of one of the wavelength bands corresponding to the red pixels and the blue pixels of the image sensor 40. The second wavelength band FR may include at least a part of the other one of the wavelength bands corresponding to the red pixels and the blue pixels of the image sensor 40. For example, the first and the second wavelength bands FL and FR may partially overlap with each other (for example, a green wavelength band).

With the first and the second wavelength bands FL and FR are separated into the wavelength bands corresponding to the red color r and the blue color b, a red color image and a blue color image can be extracted from a captured image, and thus parallax images can be obtained.

The imaging device according to the present embodiment may be configured as follows. Specifically, the according to the present embodiment includes: a memory that stores that stores information (for example, a program and various types of data); and a processor (processor including hardware) that operates based on the information stored in the memory. The processor performs a movable mask control process for controlling the movable mask 30. The movable mask control process includes setting, in the non-stereoscopic mode, the movable mask 30 to be in the first state, and setting, in the stereoscopic mode, the movable mask 30 to be in the second state.

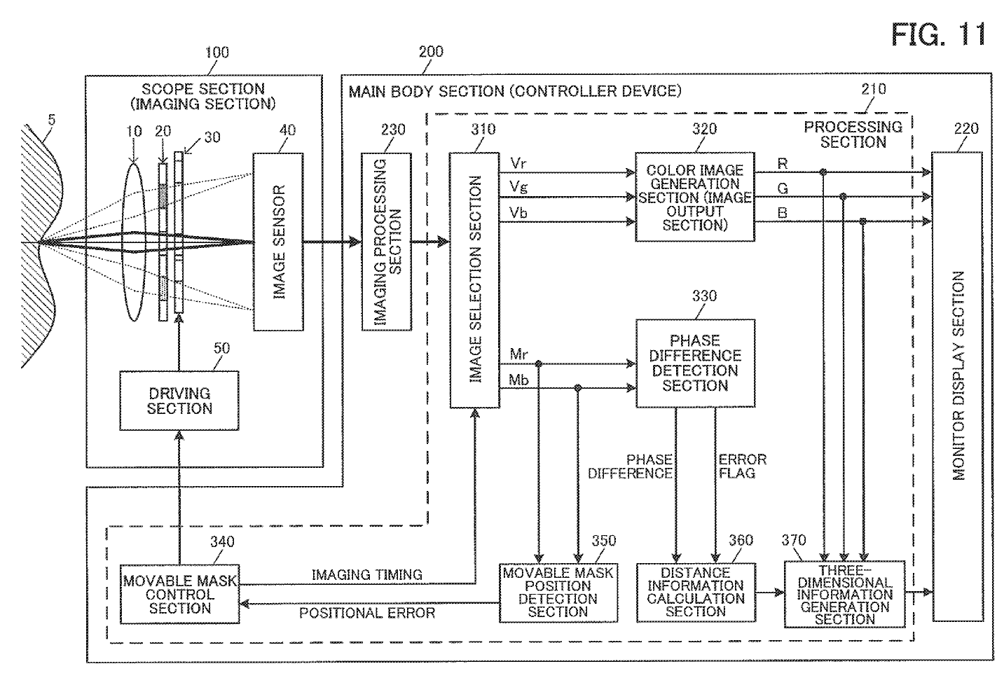

For example, the function of each section may be implemented by the processor or may be implemented by integrated hardware. For example, the processor may include hardware, and the hardware may include at least one of a circuit that processes a digital signal and a circuit that processes an analog signal. For example, the processor may include one or more circuit devices (e.g., IC), and one or more circuit elements (e.g., resistor or capacitor) that are mounted on a circuit board. The processor may be a central processing unit (CPU), for example. Note that the processor is not limited to a CPU. Various other processors such as a graphics processing unit (GPU) or a digital signal processor (DSP) may also be used. The processor may be a hardware circuit that includes an ASIC. The processor may include an amplifier circuit, a filter circuit, and the like that process an analog signal. The memory may be a semiconductor memory (e.g., SRAM or DRAM), or may be a register. The memory may be a magnetic storage device such as a hard disk drive (HDD), or may be an optical storage device such as an optical disc device. For example, the memory stores a computer-readable instruction, and the process (function) of each section of the imaging device is implemented by causing the processor to perform the instruction. As illustrated in FIG. 11 for example, sections of the imaging device include an imaging processing section 230, an image selection section 310, a color image generation section 320 (image output section), a phase difference detection section 330, a distance information calculation section 360, a movable mask position detection section 350, the movable mask control section 340, and a three-dimensional information generation section 370. The instruction may be an instruction set that is included in a program, or may be an instruction that instructs the hardware circuit included in the processor to operate.

For example, operations according to the present embodiment are implemented as follows. Specifically, the processor outputs a control signal, for setting the non-stereoscopic mode and setting the movable mask 30 to be in the first state, to a driving section 50. The driving section 50 sets the movable mask 30 to be in the first state. Specifically, the processor outputs a control signal, for setting the stereoscopic mode and setting the movable mask 30 to be in the second state, to the driving section 50. The driving section 50 sets the movable mask 30 to be in the second state.

The sections of the imaging device according to the present embodiment may be implemented as modules of a program operating on the processor. For example, the movable mask control section 340 is implemented as a movable mask control module that sets that movable mask 30 to be in the first state in the non-stereoscopic mode, and to be in the second state in the stereoscopic mode.

5. Modifications

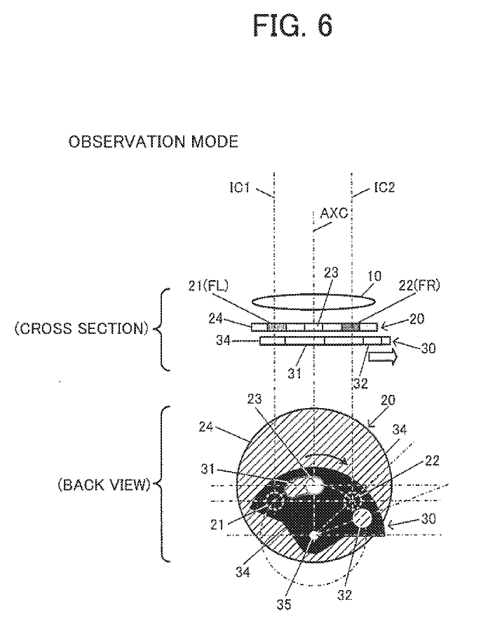

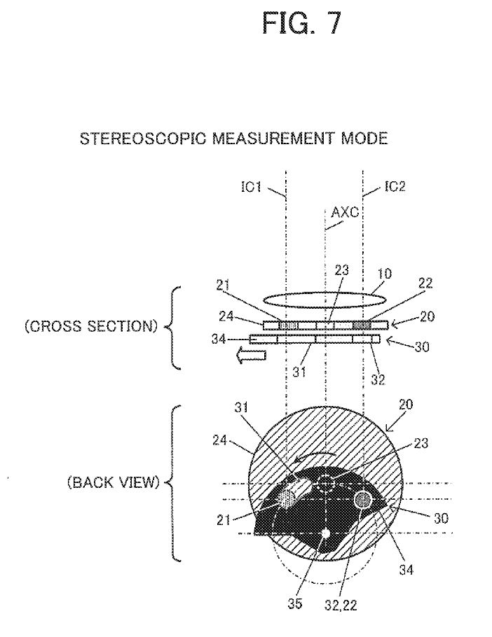

A first modification is described. The embodiment is described above with the movable mask 30 provided with the three stop holes 31, 32, and 33 as an example. However, this should not be construed in a limiting sense. For example, as illustrated in FIG. 6 and FIG. 7, the movable mask 30 may be provided with two stop holes 31 and 32.

Specifically, the movable mask 30 includes: the light shielding section 34; and the stop holes 31 and 32 provided to the light shielding section 34. For example, the stop holes 31 and 32 are in the open state (through holes), and are arranged on the same circle with the rotational shaft 35 at the center. The stop hole 31 has a shape extending in a circumference direction of the circle, to have such a shape that overlaps with the stop hole 23 of the fixed mask 20 in the observation mode and overlaps with the stop hole 21 of the fixed mask 20 in the stereoscopic measurement mode.

The fixed mask 20 includes the light shielding section 24, and the three stop holes 21, 22, and 23 provided to the light shielding section 24. The stop holes 21 and 22 are provided with the spectral filters FL and FR. The stop holes 21, 22 and 23 are arranged on the same circle with the rotational shaft 35 at the center.

In the observation mode, the stop hole 23 of the fixed mask 20 corresponding to the pupil center is in the open state through the stop hole 31 of the movable mask 30, and the stop holes 21 and 22 of the fixed mask 20 corresponding to the left and the right pupils are shielded with the light shielding section 34 of the movable mask 30, whereby a white light image is captured with a single pupil. In the stereoscopic measurement mode, the stop holes 21 and 22 of the fixed mask 20 corresponding to the left and the right pupils are in the open state with the stop holes 31 and 32 of the movable mask 30, and the stop hole 23 of the fixed mask 20 corresponding to the pupil center is shielded with the light shielding section 34 of the movable mask 30. Thus, parallax images (red image and blue image) in the color phase difference method are captured.

In this modification, an imaging device (endoscope apparatus) includes: the image sensor 40; the imaging optical system 10; the fixed mask 20; and the movable mask 30. The imaging optical system 10 forms an image of the subject 5 on the image sensor 40. The fixed mask 20 includes: the first to the third openings (the stop holes 21, 22, and 23) dividing the pupil of the imaging optical system 10; the first filter FL transmitting light in the first wavelength band; and the second filter FR transmitting light in the second wavelength band different from the first wavelength band. The movable mask 30 includes: the light shielding section 34; the fourth opening (stop hole 31) that is provided to the light shielding section 34 and corresponds to the first and the third openings (stop holes 21 and 23); and the fifth opening (stop hole 32) that is provided to the light shielding section 34 and corresponds to the second opening (stop hole 22), and is movable relative to the imaging optical system 10. The first filter FL is provided to the first opening (stop hole 21). The second filter FR is provided to the second opening (stop hole 22). The third opening (stop hole 23) is provided to the optical axis AXC of the imaging optical system 10.

Specifically, the imaging device includes the movable mask control section 340 that controls the movable mask 30. In the non-stereoscopic mode (observation mode), the movable mask control section 340 (processor) sets the movable mask 30 to be in the first state in which the light shielding section 34 overlaps with the first and the second openings (stop holes 21 and 22) and the fourth opening (stop hole 31) overlaps with the third opening (stop hole 23), as viewed in the direction along the optical axis AXC. In a stereoscopic mode (stereoscopic measurement mode), the movable mask control section 340 sets the movable mask 30 to be in the second state in which the fourth and the fifth openings (stop holes 31 and 32) overlap with the first and the second openings (stop holes 21 and 22), and the light shielding section 34 overlaps with the third opening (stop hole 23), as viewed in the direction along the optical axis AXC.

This configuration can also achieve switching between the observation mode and the stereoscopic measurement mode, simultaneous acquisition of parallax images in the stereoscopic measurement mode, high speed mode switching, a simplified driving mechanism for the movable mask 30, and reduction of a risk of failure and error due to the mode switching, and can ensure the baseline length for the stereoscopic measurement.

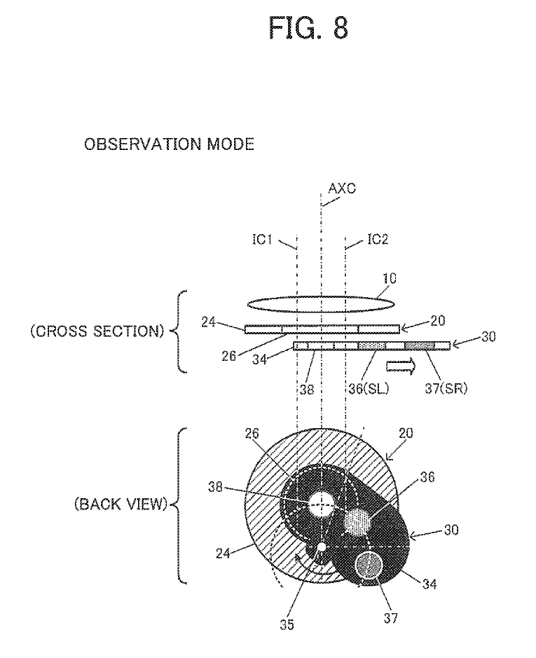

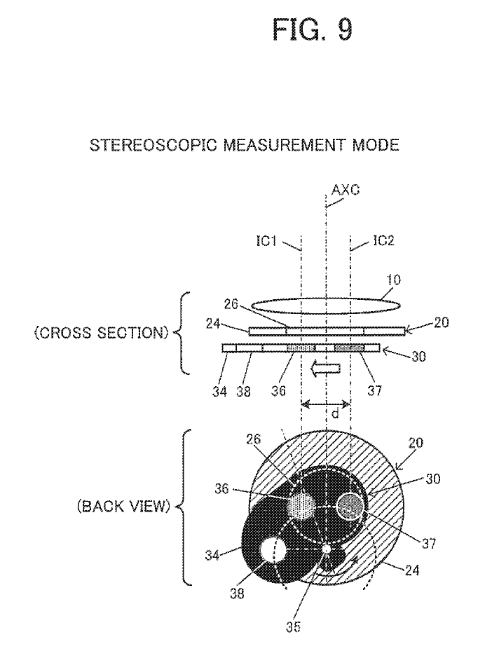

Next, a second modification is described. The embodiment is described above with a case where the pupil divided with the fixed mask 20 as an example. However, this should not be construed in a limiting sense. For example, as illustrated in FIG. 8 and FIG. 9, the pupil may be divided with the movable mask 30.

Specifically, the fixed mask 20 includes: the light shielding section 24; and a single stop hole 26 provided to the light shielding section 24. The stop hole 26 has a larger size (diameter of the circle) than the stop holes 36, 37, and 38 of the movable mask 30, and has a size large enough to at least cover the stop holes 36 and 37 of the movable mask 30.

The movable mask 30 includes: the light shielding section 34; and the stop holes 36, 37, and 38 provided to the light shielding section 34. The spectral filters SL and SR are provided to the stop holes 36 and 37. The spectral characteristics of the spectral filters SL and SR are the same as the spectral characteristics FL and FR in FIG. 5. The stop hole 38 is in the open state (through hole). The stop holes 36, 37, and 38 are on the same circle with the rotational shaft 35 at the center.

In the observation mode, the stop hole 38 of the movable mask 30 moves to the pupil center to overlap with the stop hole 26 of the fixed mask 20 to be in the opened state. The stop holes 36 and 37 of the movable mask 30 are shielded with the shielding section 24 of the fixed mask 20, whereby a white light image is captured with a single optical system. In the stereoscopic measurement mode, the stop holes 36 and 37 of the movable mask 30 overlap with the stop hole 26 of the fixed mask 20 to be in the opened state. The stop hole 26 of the movable mask 30 is shielded with the light shielding section 24 of the movable mask 30. Thus, parallax images (red image and blue image) in the color phase difference method are captured.

In this modification, an imaging device (endoscope apparatus) includes: the image sensor 40; the imaging optical system 10; the movable mask 30; and the fixed mask 20. The imaging optical system 10 forms an image of the subject 5 on the image sensor 40. The movable mask 30 includes the first to the third openings (stop holes 36, 37, and 38), and is movable relative to the imaging optical system 10. The fixed mask 20 has a fourth opening (stop hole 26) provided to the optical axis AXC of the imaging optical system 10. The movable mask 30 includes: the first filter SL, transmitting light in a first wavelength band, provided to the first opening (stop hole 36); and the second filter FR, transmitting light in a second wavelength band different from the first wavelength band, provided to the second opening (stop hole 37). The fourth opening (stop hole 26) is an opening with a size larger than the distance (baseline length d) between the first and the second openings (stop holes 36 and 37).

Specifically, the imaging device includes the movable mask control section 340 that controls the movable mask 30. In the non-stereoscopic mode (observation mode), the movable mask control section 340 (processor) sets the movable mask 30 to be in the first state in which the first and the second openings (stop holes 36 and 37) do not overlap with the fourth opening (stop hole 26) and the third opening (stop hole 38) is inserted on the optical axis AXC, as viewed in the direction along the optical axis AXC. In a stereoscopic mode (stereoscopic measurement mode), the movable mask control section 340 sets the movable mask 30 to be in the second state in which the first and the second openings (stop holes 36 and 37) overlap with the fourth opening (stop hole 26), and the third opening (stop hole 38) does not overlap with the fourth opening (stop hole 26), as viewed in the direction along the optical axis AXC.

This configuration can also achieve switching between the observation mode and the stereoscopic measurement mode, simultaneous acquisition of parallax images in the stereoscopic measurement mode, high speed mode switching, a simplified driving mechanism for the movable mask 30, and reduction of a risk of failure and error due to the mode switching, and can ensure the baseline length for the stereoscopic measurement.

6. Principle of Stereoscopic Three-Dimensional Measurement

The principle of the stereoscopic measurement in the stereoscopic measurement mode is described. As illustrated in FIG. 10, the optical paths for the left eye and the right eye are each independently formed. Reflected light from the subject 5 passes through these optical paths so that the subject image is formed on the image sensor surface (light receiving surface). A coordinate system X, Y, Z in the three-dimensional space is defined as follows. Specifically, an X axis and a Y axis orthogonal to the X axis are set along the image sensor surface. A Z axis, toward the subject, is set to be in a direction that is orthogonal to the image sensor surface, and parallel to the optical axis AXC. The Z axis, the X axis, and the Y axis intersect at the zero point. The Y axis is omitted for the sake of illustration.

Here, the distance between the imaging lens 10 and the image sensor surface is defined as b, and the distance between the imaging lens 10 and a given point Q(x,z) of the subject 5 is defined as z. The centerlines IC1 and IC2 of the pupils are separated from the Z axis by the same distance d/2. Thus, the baseline length for the stereoscopic measurement is d. An X coordinate of a corresponding point, corresponding to the given point Q(x,y) of the subject 5, as a part of an image formed on the image sensor surface with the imaging lens 10 is XL. An X coordinate of the corresponding point, corresponding to the given point Q(x,y) of the subject 5, as a part of the image formed on the image sensor surface with the imaging lens 10 is XR. The following Formula (3) can be obtained based on a similarity relation among a plurality of partial right angle triangles formed within a triangle defined by the given point Q(x,z) and the coordinates XL and XR.

$$\begin{matrix}[ Formula 3 ] & \\ \frac{z}{ b} = \frac{\vert x + d / 2 \vert}{\vert XL + d / 2 \vert} = \frac{\vert x - d / 2 \vert}{\vert XR - d / 2 \vert} & ( 3 )\end{matrix}$$

The following Formulae (4) and (5) hold true.

[Formula 4]

x+d/2>0 when XL+d/2<0

x+d/2<0 when XL+d/2>0 (4)

[Formula 5]

x−d/2>0 when XR−d/2<0

x−d/2<0 when XR−d/2>0 (5)

Thus, the absolute value in Formula (3) described above can be normal values as in the following Formula (6).

$$\begin{matrix}[ Formula 6 ] & \\ \frac{z}{ b} = - \frac{x + d / 2}{XL + d / 2} = - \frac{x - d / 2}{XR - d / 2} & ( 6 )\end{matrix}$$

Formula (6) described above can be solved for x as in the Formula (7).

$$\begin{matrix}[ Formula 7 ] & \\ x = \frac{- d}{2} \cdot \frac{XR + XL}{XR - XL - d} & ( 7 )\end{matrix}$$

The following Formula (8) for obtaining z can be obtained by substituting x in Formula (7) described above into Formula (6) described above.

$$\begin{matrix}[ Formula 8 ] & \\ z = \frac{d}{( XR - XL - d )} \cdot b & ( 8 )\end{matrix}$$

Here, d and b are known setting values, and the unknown values XL and XR are obtained as follows. Specifically, the position XL on the image sensor surface is used as a reference (the pixel position in the left image is regarded as XL), and the position XR corresponding to the position XL is detected with matching processing (correlation calculation). The subject shape can be measured by calculating the distance z for each position XL. Some distances z might be unobtainable due to matching failure. Such distances z may be obtained by interpolation using the distances z obtained for the surrounding pixels or by other like method, for example.

7. Endoscope Apparatus

FIG. 11 illustrates an example of a configuration of an endoscope apparatus (an imaging device in a broad sense) according to the present embodiment. The scope section 100 (image capturing section) includes a scope section 100 (image capturing section) and a main body section 200 (controller device). The scope section 100 includes the imaging optical system 10, the fixed mask 20, the movable mask 30, the image sensor 40, and the driving section 50. The main body section 200 includes a processing section 210, a monitor display section 220, and the imaging processing section 230. The processing section 210 includes the image selection section 310 (image frame selection unit), the color image generation section 320 (image output section), the phase difference detection section 330, the movable mask control section 340 (movable mask driving controller section), the movable mask position detection section 350, the distance information calculation section 360, and the three-dimensional information generation section 370.

The main body section 200 may further include unillustrated elements such as an operation section for operating the main body section 200 and an interface section for connecting with external devices. The scope section 100 may further include unillustrated elements such as, for example, an operation section for operating the scope section 100, a treatment section, and an illumination section (a light source, a lens, and the like).

The endoscope apparatus may be what is known as a video scope (an endoscope apparatus incorporating an image sensor) for industrial and medical use. The present invention can be applied to a flexible endoscope with the scope section 100 that is flexible and to a rigid endoscope with the scope section 100 that is in a form of a stick. For example, a flexible endoscope for industrial use includes the main body section 200 and the image capturing section 110 serving as a portable device that can be carried around. The flexible endoscope is used for inspection in manufacturing and maintenance processes for industrial products, in a maintenance process for buildings and pipes, and in other like situations.

The driving section 50 drives the movable mask 30 based on the control signal from the movable mask control section 340, to switch between the first state (observation mode) and the second state (stereoscopic measurement mode). For example, the driving section 50 includes an actuator including a piezoelectric element and a magnet mechanism.

The imaging processing section 230 executes an imaging process on a signal from the image sensor 40, and outputs a captured image (such as a Bayer image, for example). For example, a correlative double sampling process, a gain control process, an A/D conversion process, gamma correction, color correction, noise reduction, and the like are executed. For example, the imaging processing section 230 may include a discrete IC such as an ASIC, or may be incorporated in the image sensor 40 (sensor chip) and the processing section 210.

The monitor display section 220 displays an image captured by the scope section 100, three-dimensional shape information on the subject 5, or the like. For example, the monitor display section 220 includes a liquid crystal display, an Electro-Luminescence (EL) display, and the like.

An operation of the endoscope apparatus is described below. The movable mask control section 340 controls the driving section 50, and thus switches the position of the movable mask 30. When the movable mask control section 340 sets the movable mask 30 to be in the observation mode, an image of the subject 5 is formed on the image sensor 40 with reflected light from the subject 5 that has passed through the pupil-center optical path. The imaging processing section 230 reads out pixel values of the image formed on the image sensor 40, performs the A/D conversion or the like, and outputs resultant image data to the image selection section 310.

The image selection section 310 detects that the movable mask 30 is in the state corresponding to the observation mode based on the control signal from the movable mask control section 340, and outputs {Vr, Vg, Vb}, selected from the captured image, to the color image generation section 320. The color image generation section 320 performs demosaicing process (process for generating an RGB image from a Bayer image) and various image processes, and outputs the resultant three-board RGB primary color image to the monitor display section 220. The monitor display section 220 displays this color image.

When the movable mask control section 340 sets the movable mask 30 to be in the stereoscopic measurement mode, images are simultaneously formed on the image sensor 40 based on the reflected light from the subject 5, through the left-pupil optical path and the right-pupil optical path. The imaging processing section 230 reads out pixel values of the image formed on the image sensor 40, performs the A/D conversion or the like, and outputs resultant image data to the image selection section 310.

The image selection section 310 detects that the movable mask 30 is in the state corresponding to the stereoscopic measurement mode based on the control signal from the movable mask control section 340, and outputs {Mr,Mb }, selected from the captured image, to the phase difference detection section 330. The phase difference detection section 330 executes a matching process on the two separate images Mr and Mb, to detect a phase difference (phase shift) for each pixel. The phase difference detection section 330 determines whether the detected phase difference is reliable, and outputs an error flag for each pixel determined to have an unreliable phase difference. Various matching evaluation methods for obtaining the amount of difference (phase difference) between two similar waveforms have conventionally been proposed, and thus can be used as appropriate. The proposed methods include normalized correlation calculation such as Zero-mean Normalized Cross-Correlation (ZNCC), and Sum of Absolute Difference (SAD) based on the sum of absolute differences between the waveforms.

Furthermore, parallax images Vr and Mr may be used for detecting phase shift (phase difference), but this method involves time division, which is negatively affected by movement of the subject and/or the imaging system. Furthermore, parallax images Vr and Mr may be used for detecting phase shift (phase difference), but this method involves time division, which is negatively affected by movement of the subject and/or the imaging system.

The phase difference detection section 330 outputs the phase difference information thus detected, and the error flag to the distance information calculation section 360. The distance information calculation section 360 calculates the distance information (for example, the distance z in FIG. 10) on the subject 5 for each pixel, and outputs the resultant distance information to the three-dimensional information generation section 370. For example, the pixel provided with the error flag may be regarded as a flat portion of the subject 5 (an area with a small amount of edge components), and interpolation may be performed for such pixel based on the distance information on surrounding pixels. The three-dimensional information generation section 370 generates three-dimensional information from the distance information (or from the distance information and the RGB image from the color image generation section 320). The three-dimensional information may be various types of information including a Z value map (distance map), polygon, and a pseudo-three-dimensional display image (with shape emphasized by shading or the like, for example). The three-dimensional information generation section 370 generates a three-dimensional image and three-dimensional data generated, or a display image obtained by superimposing the observation image on the image as appropriate, and outputs the resultant image and/or data to the monitor display section 220. The monitor display section 220 displays the three-dimensional information.

The movable mask position detection section 350 detects whether the movable mask 30 is at the position corresponding to the observation mode or at the position corresponding to the stereoscopic measurement mode by using the images {Mr,Mb } obtained in the stereoscopic measurement mode. When the movable mask 30 is in the state not matching the mode, a position error flag is output to the movable mask control section 340. Upon receiving the position error flag, the movable mask control section 340 corrects the movable mask 30 to be in the correct state (state corresponding to the image selection). For example, when the images {Mr,Mb } are determined to have no color shift even though the movable mask control section 340 is outputting the control signal for achieving the stereoscopic measurement mode, the movable mask 30 is actually at the position for the observation mode. In such a case, the correction is performed in such a manner that the position of the movable mask 30 matches the position indicated by the control signal. When the correction operation cannot achieve the correct state, some sort of failure is determined to have occurred, and thus the function of the entire system is stopped. For example, whether the movable mask 30 is at the position corresponding to the observation mode or is at the position corresponding to the stereoscopic measurement mode is detected or determined as follows.

Specifically, whether a position error has occurred is determined by matching the level (average level or the like) between determination areas in the image Mr and the image Mb, and then performing determination on a position error based on the sum of absolute difference between the image Mr and the image Mb (first method), determination based on correlation coefficients of the image Mr and the image Mb (second method), and the like.

In the first method, an absolute difference value between pixel values at each pixel is obtained, and the absolute values of all the pixels or a group of some of the pixels are integrated. When the resultant value exceeds a predetermined threshold, the image is determined to be that in the stereoscopic measurement mode. On the other hand, when the resultant value does not exceed the predetermined threshold, the image is determined to be that in the observation mode. In the stereoscopic measurement mode, basically, the image Mr and the image Mb have color misregistration, resulting in a predetermined difference value. Thus, the first method is performed based on this value.

In the second method, the correlation coefficient between the image Mr and the image Mb within a predetermined range is calculated. When the result of the calculation does not exceed a predetermined threshold, the image is determined as that in the stereoscopic measurement mode. When the result exceeds the predetermined threshold, the image is determined to be that in the observation mode. This method is based on the fact that the image obtained in the stereoscopic measurement mode has a small relative coefficient because the image Mr and the image Mb basically have color misregistration, and that the image Mr and the image Mb in the observation mode substantially match, and thus have a large relative coefficient.

8. Mode Switching Sequence

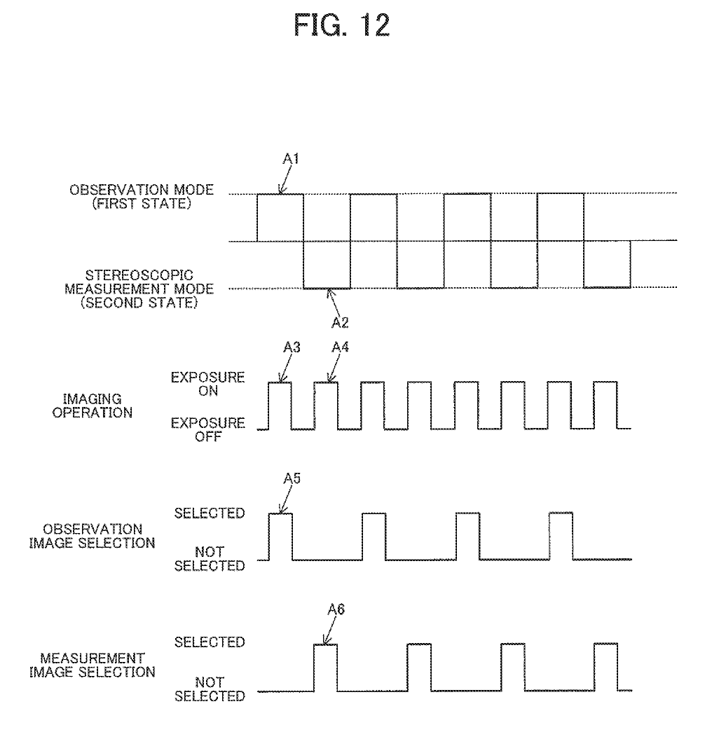

FIG. 12 illustrates a sequence for switching from the observation mode to the stereoscopic measurement mode in moving image capturing (operation timing chart).

With the stereoscopic measurement mode described above, accurate real time stereoscopic measurement can be performed even on a moving subject. However, the image obtained has color misregistration and thus cannot be used as a high level observation image This can be overcome through high speed switching between the observation mode and the stereoscopic measurement mode. Thus, the stereoscopic measurement can be executed while displaying the observation image in substantially real time.

As illustrated in FIG. 12, switching of the state of the movable mask 30, an image capturing timing, and selection of the captured image are interlocked. As indicated by A1 and A2, the mask state corresponding to the observation mode and the mask state corresponding to the stereoscopic measurement mode are alternately achieved. As indicated by A3 and A4, an image is captured each time the mask state changes. As indicated by A5, the image captured with the image sensor 40 exposed in the mask state corresponding to the observation mode is selected as an observation image. As indicated by A6, an image captured with the image sensor 40 exposed in the stereoscopic measurement mode is selected as a measurement image.

With the observation mode and the stereoscopic measurement mode thus alternately repeated, the observation image and the measurement image can be contiguously obtained in substantially real time. Thus, the observing and the measurement can both be implemented even when the subject 5 is moving. When the image obtained in the observation mode is displayed with measurement information overlaid as appropriate, useful information can be provided so that the user can perform visual inspection and quantitative inspection at the same time.

In the present embodiment, in a first frame (A1 in FIG. 14), the movable mask control section 340 sets the non-stereoscopic mode (observation mode) and a first captured image (observation image) is obtained with the image sensor 40 (A3). Then, in a second frame (A2) subsequent to the first frame, the movable mask control section 340 sets the stereoscopic mode (stereoscopic measurement mode), and a second captured image (measurement image) is obtained with the image sensor 40 (A4).

Specifically, the imaging device (endoscope apparatus) alternately repeats the first frame (A1) and the second frame (A2) when the moving image is being captured. Thus, an operation that is the same as that in the first frame is performed in a third frame subsequent to the second frame.

More specifically, the imaging device includes: the image output section (the color image generation section 320, which is a processor) that outputs a moving image for observation; and the phase difference detection section 330 (processor) that detects a phase difference between the image (blue image Mb) corresponding to the first wavelength band and the image (red image Mr) corresponding to the second wavelength band based on the second captured image in the moving image.

With the moving image captured by alternately repeating the image capturing in the observation mode and the image capturing in the stereoscopic measurement mode, the real time stereoscopic measurement can be performed for the subject 5 while performing the observation with a normal image obtained with a single optical system. The configuration according to the present embodiment features the movable mask 30 and the fixed mask 20 suitable for high speed switching, and thus is suitable for the real time measurement.

In the present embodiment, the imaging device includes the movable mask position detection section 350. The movable mask position detection section 350 (processor) detects whether the movable mask 30 is set to be in the second state in the stereoscopic mode, based on a similarity (based on the sum of absolute differences, the correlation coefficient, or the like described above with reference to FIG. 13) between the image (blue image Mb) corresponding to the first wavelength band and the image (red image Mr) corresponding to the second wavelength band in the image captured in the stereoscopic mode.

When it is determined that the movable mask 30 is set in the first state in the stereoscopic mode, the movable mask control section 340 corrects the movable mask 30 so that the state and the mode match.

The mechanical movable member such as the movable mask 30 might not actually operate as indicated by the control due to factors such as operation failure, for example. When such an error occurs, an image with color misregistration might be displayed as the observation image, or appropriate stereoscopic measurement might not be achievable. In view of this, in the present embodiment, whether the mask state matches the mode can be determined based on the similarity between the parallax images. Thus, the mask state can be corrected to match the mode based on the result of the determination. In the observation mode, a red image and a blue image are captured with a single pupil, and thus involve no phase difference and have high similarity. Thus, the movable mask 30 can be determined to be erroneously at the position corresponding to the observation mode, when a red image and a blue image having high similarity are obtained in the stereoscopic measurement mode.

The embodiments and the modifications thereof according to the present invention are described. However, the present invention is not limited the embodiments and the modifications only, and the present invention can be implemented with the elements modified without departing from the gist of the invention. The plurality of elements disclosed in the embodiments and the modifications may be combined as appropriate to implement the invention in various ways. For example, some of all the elements described in the embodiments and the modifications may be deleted. Furthermore, elements in different embodiments and modifications may be combined as appropriate. Thus, various modification and application can be made without departing from the gist of the present invention. The terms (observation mode, stereoscopic measurement mode, and the like) that is at least once written as the other term with a broader concept or a narrower concept (non-stereoscopic mode, stereoscopic measurement mode, and the like) can be replaced with the other terms in any portion of the specification or figures.

What is claimed is:

1. An imaging device comprising: an image sensor;
an imaging optical device forming an image of a subject on the image sensor;
a fixed mask including first to third openings dividing a pupil of the imaging optical system, a first filter transmitting light in a first wavelength band, and a second filter transmitting light in a second wavelength band different from the first wavelength band;
a movable mask including a light shielding section and fourth to sixth openings that are provided on the light shielding section and correspond to the first to the third openings, the movable mask being movable relative to the imaging optical system,
the first filter is provided to the first opening,
the second filter being provided to the second opening,
the third opening being provided on an optical axis of the imaging optical device.

2. The imaging device as defined in claim 1,
further comprising a processor comprising hardware, the processor being configured to implement:
a movable mask control process that controls the movable mask,
the processor implementing the movable mask control process including:
setting, in a non-stereoscopic mode, the movable mask to be in a first state in which the light shielding section overlaps with the first and the second openings, and the sixth opening overlaps with the third opening, as viewed in a direction of the optical axis; and
setting, in stereoscopic mode, the movable mask to be in a second state in which the fourth and the fifth openings overlap with the first and the second openings, and the light shielding section overlaps with the third opening, as viewed in the direction of the optical axis.

3. The imaging device as defined in claim 2,
the processor implementing the movable mask control process including setting the non-stereoscopic mode, the image sensor capturing a first captured image, in a first frame, and
the processing implementing the movable mask control process including setting the stereoscopic mode, the image sensor capturing a second captured image, in a second frame subsequent to the first frame.

4. The imaging device as defined in claim 3,
the first frame and the second frame being alternately repeated when a moving image is captured.

5. The imaging device as defined in claim 4,
the processor being configured to implement:
an image output process that outputs an observation moving image based on the first captured image in the moving image; and
a phase difference detection process that detects a phase difference between an image corresponding to the first wavelength band and an image corresponding to the second wavelength band, based on the second captured image in the moving image.

6. The imaging device as defined in claim 2,
the processor being configured to implement:
a movable mask detection process that detects whether the movable mask is set to be in the second state in the stereoscopic mode, based on a similarity between an image corresponding to the first wavelength band and an image corresponding to the second wavelength band that are in an image captured in the stereoscopic mode.

7. The imaging device as defined in claim 6,
the processor being configured to implement:
the movable mask control process including
performing correction, when the movable mask is detected to be in the first state in the stereoscopic mode, in such a manner that correct relationship between a state of the movable mask and the mode match.

8. The imaging device as defined in claim 1,
further comprising a processor comprising hardware, the processor being configured to implement
a phase difference detection process that detects a phase difference between an image corresponding to the first wavelength band and an image corresponding to the second wavelength band, based on an image captured under a condition that the movable mask is set to be in a state in which the light shielding section overlaps with the first and the second openings, and the sixth opening overlaps with the third opening, as viewed in the direction of the optical axis.

9. The imaging device as defined in claim 1,
the captured image obtained by the image sensor including images of a red color, a green color, and a blue color,
the first wavelength band corresponding to one of the red color and the blue color,
the second wavelength band being a wavelength band corresponding to another one of the red color and the blue color.

10. An imaging device comprising: an image sensor;
an imaging optical device forming an image of a subject on the image sensor;
a fixed mask including first to third openings dividing a pupil of the imaging optical system, a first filter transmitting light in a first wavelength band, and a second filter transmitting light in a second wavelength band different from the first wavelength band;
a movable mask including a light shielding section, a fourth opening that is provided on the light shielding section and corresponds to the first to the third openings, and a fifth opening that is provided on the light shielding section and corresponds to the second opening, the movable mask being movable relative to the imaging optical system,
the first filter is provided to the first opening,
the second filter being provided to the second opening,
the third opening being provided on an optical axis of the imaging optical device.

11. The imaging device as defined in claim 10,
further comprising a processor comprising hardware, the processor being configured to implement
a movable mask control process that controls the movable mask,
the processor implementing the movable mask control process including:
setting, in a non-stereoscopic mode, the movable mask to be in a first state in which the light shielding section overlaps with the first and the second openings, and the fourth opening overlaps with the third opening, as viewed in a direction of the optical axis; and
setting, in stereoscopic mode, the movable mask to be in a second state in which the fourth and the fifth openings overlap with the first and the second openings, and the light shielding section overlaps with the third opening, as viewed in the direction of the optical axis.

12. An imaging device comprising: an image sensor;
an imaging optical device forming an image of a subject on the image sensor;
a movable mask including first to the third openings, the movable mask being movable relative to the imaging optical system;
a fixed mask including a fourth opening provided on an optical axis of the imaging optical device; and
a processor being configured to implement a movable mask control process for controlling the movable mask,
the movable mask includes:
a first filter being provided to the first opening and transmitting light in a first wavelength band; and
a second filter being provided to the second opening and transmitting light in a second wavelength band different from the first wavelength band,
the fourth opening has
a size larger than a distance between the first and the second openings,
the processor implementing the movable mask control process including:
setting, in a non-stereoscopic mode, the movable mask to be in a first state in which the first and the second openings do not overlap with the fourth opening and the third opening is on the optical axis, as viewed in a direction of the optical axis; and
setting, in a stereoscopic mode, the movable mask to be in a second state in which the first and the second openings overlap with the fourth opening, and the third opening does not overlap with the fourth opening, as viewed in the direction of the optical axis.

13. An endoscope apparatus comprising the imaging device as defined in claim 1.

14. An endoscope apparatus comprising the imaging device as defined in claim 10.

15. An endoscope apparatus comprising the imaging device as defined in claim 12.

16. An imaging method comprising: setting, in a non-stereoscopic mode, a movable mask, including a light shielding section and fourth to sixth openings that are provided to the light shielding section and correspond to first to third openings of a fixed mask, to be in a first state, in such a manner that the light shielding section overlaps with the first opening provided with a first filter transmitting light in a first wavelength band and the second opening provided with a second filter transmitting light in a second wavelength band different from the first wavelength band, and that the sixth opening overlaps with the third opening, as viewed in a direction of an optical axis of an imaging optical device; and
setting, in a stereoscopic mode, the movable mask to be in a second state in such a manner that the fourth and the fifth openings overlap with the first and the second openings and the light shielding section overlaps with the third opening, as viewed in the direction of the optical axis.
